SEQUENCE LISTING (47 sequences)

SEQ ID NO: 1 - protein, 13 aa, Artificial Sequence
  Description of Artificial Sequence Synthetic peptide
XXVXGWGXVPSNG

SEQ ID NO: 2 - protein, 11 aa, Artificial Sequence
  Description of Artificial Sequence Synthetic peptide
TPFTDXXTEKL

SEQ ID NO: 3 - protein, 8 aa, Artificial Sequence
  Description of Artificial Sequence Synthetic peptide
ELAKKXGX

SEQ ID NO: 4 - protein, 13 aa, Artificial Sequence
  Description of Artificial Sequence Synthetic peptide
FIKAXXXGNXXDA

SEQ ID NO: 5 - protein, 13 aa, Artificial Sequence
  Description of Artificial Sequence Synthetic peptide
TLKAXXXGNXXDA

SEQ ID NO: 6 - protein, 16 aa, Artificial Sequence
  Description of Artificial Sequence Synthetic peptide
XLKXXXAXXLGNXXDA

SEQ ID NO: 7 - DNA, 27 bp, Artificial Sequence
  Description of Artificial Sequence Synthetic primer
cccgaattcatgaaggtagaggtgctg

SEQ ID NO: 8 - DNA, 30 bp, Artificial Sequence
  Description of Artificial Sequence Synthetic primer
cccaagcttttagatggtgtactcgtggcc

SEQ ID NO: 9 - DNA, 27 bp, Artificial Sequence
  Description of Artificial Sequence Synthetic primer
cccggtaccgggcgcgtaaaaggatct

SEQ ID NO: 10 - DNA, 27 bp, Artificial Sequence
  Description of Artificial Sequence Synthetic primer
cccggtacctcgactgagcctttcgtt

SEQ ID NO: 11 - DNA, 27 bp, Artificial Sequence
  Description of Artificial Sequence Synthetic primer
gcgggtacctgatctgatccttcaact

SEQ ID NO: 12 - DNA, 27 bp, Artificial Sequence
  Description of Artificial Sequence Synthetic primer
gcgggtaccctcagcaaaagttcgatt

SEQ ID NO: 13 - DNA, 18 bp, Artificial Sequence
  Description of Artificial Sequence Synthetic primer
gtcccagtggtggtgggt

SEQ ID NO: 14 - DNA, 18 bp, Artificial Sequence
  Description of Artificial Sequence Synthetic primer
acctgtgaacacggcagg

SEQ ID NO: 15 - DNA, 18 bp, Artificial Sequence
  Description of Artificial Sequence Synthetic primer
caggcacttgacgttcag

SEQ ID NO: 16 - DNA, 36 bp, Artificial Sequence
  Description of Artificial Sequence Synthetic primer
acccaccaccactgggactgtgctggcgggaatgag

SEQ ID NO: 17 - DNA, 39 bp, Artificial Sequence
  Description of Artificial Sequence Synthetic primer
cctgccgtgttcacaggttgtaccttgtttgtggctggc

SEQ ID NO: 18 - DNA, 63 bp, Artificial Sequence
  Description of Artificial Sequence Synthetic primer
ctgaacgtcaagtgcctgtataccgggccgtgccacactacagaccacatttgttacttcgtg

SEQ ID NO: 19 - DNA, 18 bp, Artificial Sequence
  Description of Artificial Sequence Synthetic primer
cagggcaggcagcacctc

SEQ ID NO: 20 - DNA, 75 bp, Artificial Sequence
  Description of Artificial Sequence Synthetic primer
gaggtgctgcctgccctgnnsnnsgttnnsgggtggggcnnsgtaccttccaacgggtacctggtcattgatgat

SEQ ID NO: 21 - DNA, 18 bp, Artificial Sequence
  Description of Artificial Sequence Synthetic primer
atccacaatggcagcctc

SEQ ID NO: 22 - DNA, 69 bp, Artificial Sequence
  Description of Artificial Sequence Synthetic primer
gaggctgccattgtggatactccatttacggatnnsnnsactgaaaagttagtggacgcggcgagaaag

SEQ ID NO: 23 - DNA, 18 bp, Artificial Sequence
  Description of Artificial Sequence Synthetic primer
gatacggtcgtcaccccc

SEQ ID NO: 24 - DNA, 60 bp, Artificial Sequence
  Description of Artificial Sequence Synthetic primer
gggggtgacgaccgtatcgagctcgccaagaaannsgggnnsggggccctgactcacaag

SEQ ID NO: 25 - DNA, 18 bp, Artificial Sequence
  Description of Artificial Sequence Synthetic primer
acctgtgaacacggcagg

SEQ ID NO: 26 - DNA, 78 bp, Artificial Sequence
  Description of Artificial Sequence Synthetic primer
cctgccgtgttcacaggttgttttattaaagcgnnsnnsnnsggcaatnnsnnsgacgcaactgcggatgagatgtgt

SEQ ID NO: 27 - DNA, 78 bp, Artificial Sequence
  Description of Artificial Sequence Synthetic primer
cctgccgtgttcacaggttgtaccttgaaagcgnnsnnsnnsggcaatnnsnnsgacgcaactgcggatgagatgtgt

SEQ ID NO: 28 - DNA, 87 bp, Artificial Sequence
  Description of Artificial Sequence Synthetic primer
cctgccgtgttcacaggttgtnnsttgaaannsnnsnnsgccnnsnnsttgggcaatnnsnnsgacgcaactgcggatgagatgtgt

SEQ ID NO: 29 - protein, 198 aa, Artificial Sequence
  Description of Artificial Sequence Synthetic polypeptide
MKVEVLPALTTVAGWGEVPSNGYLVIDDGTKEAAIVDTPFTAIDTEKLADAAGKHGVEPTTVSATHWHWDCDGGNEELVKLESGPEVYGGDGRINLAKEKWLRALAHKIAHLSTLLVESLSVKCPYPGPGHSTDDICYFASKPGGPEPHPVFRGCFIKPCALPEVLDPTADEMAKALLEVLGRLPLDTRVYYGHEYTI

SEQ ID NO: 30 - protein, 15 aa, Artificial Sequence
  Description of Artificial Sequence Synthetic peptide
FEEVNGWGVVPKHGL

SEQ ID NO: 31 - protein, 15 aa, Artificial Sequence
  Description of Artificial Sequence Synthetic peptide
LAEIEGWGMVPSNGM

SEQ ID NO: 32 - protein, 14 aa, Artificial Sequence
  Description of Artificial Sequence Synthetic peptide
LGSFNGEAVPSNGL

SEQ ID NO: 33 - protein, 7 aa, Artificial Sequence
  Description of Artificial Sequence Synthetic peptide
LTDNYMY

SEQ ID NO: 34 - protein, 13 aa, Artificial Sequence
  Description of Artificial Sequence Synthetic peptide
DTPFTAKDTEKLV

SEQ ID NO: 35 - protein, 13 aa, Artificial Sequence
  Description of Artificial Sequence Synthetic peptide
DTPINDAQTETLV

SEQ ID NO: 36 - protein, 13 aa, Artificial Sequence
  Description of Artificial Sequence Synthetic peptide
DSSWDDKLTKELI

SEQ ID NO: 37 - protein, 9 aa, Artificial Sequence
  Description of Artificial Sequence Synthetic peptide
DPVQPQKVV

SEQ ID NO: 38 - protein, 10 aa, Artificial Sequence
  Description of Artificial Sequence Synthetic peptide
NELLKKDGKV

SEQ ID NO: 39 - protein, 10 aa, Artificial Sequence
  Description of Artificial Sequence Synthetic peptide
IDLAKEKGLP

SEQ ID NO: 40 - protein, 10 aa, Artificial Sequence
  Description of Artificial Sequence Synthetic peptide
AELAKKNGYE

SEQ ID NO: 41 - protein, 15 aa, Artificial Sequence
  Description of Artificial Sequence Synthetic peptide
CFIKPYGLGNLGDAN

SEQ ID NO: 42 - protein, 18 aa, Artificial Sequence
  Description of Artificial Sequence Synthetic peptide
CMLKDNQATSIGNISDAD

SEQ ID NO: 43 - protein, 18 aa, Artificial Sequence
  Description of Artificial Sequence Synthetic peptide
CLVKSTSAKDLGNVADAY

SEQ ID NO: 44 - protein, 15 aa, Artificial Sequence
  Description of Artificial Sequence Synthetic peptide
DTLFVAGCGKFYEGT

SEQ ID NO: 45 - protein, 209 aa, Artificial Sequence
  Description of Artificial Sequence Synthetic polypeptide
MKVEVLPALTDNYMYLVIDDETKEAAIVDPVQPQKVVDAARKHGVKLTTVLTTHHHWDHAGGNEKLVKLESGLKVYGGDDRIGALTHKITHLSTLQVGSLNVKCLATPCHTSGHICYFVSKPGGSEPPAVFTGDTLFVAGCGKFYEGTADEMCKALLEVLGRLPPDTRVYCGHEYTINNLKFARHVEPGNAAIREKLAWAKEKYSIGEP

SEQ ID NO: 46 - protein, 6 aa, Artificial Sequence
  Description of Artificial Sequence Synthetic peptide
FKMPRD

SEQ ID NO: 47 - protein, 208 aa, Artificial Sequence
  Description of Artificial Sequence Synthetic polypeptide
EGVYVHTSFEEVNGWGVVPKHGLVVLVNAEAYLIDTPFTAKDTEKLVTWFVERGYKIKGSISSHFHSDSTGGIEWLNSRSIPTYASELTNELLKKDGKVQATNSFSGVNYWLVKNKIEVFYPGPGHTPDNVVVWLPERKILFGGCFIKPYGLGNLGDANIEAWPKSAKLLKSKYGKAKLVVPSHSEVGDASLLKLTLEQAVKGLNESK

(19) United States
(12) Patent Application Publication
(10) Pub. No.: US 2010/0204450 A1
(43) Pub. Date: Aug. 12, 2010

(54) Preparation Method for a Protein With New Function Through Simultaneous Incorporation of Functional Elements

(76) Inventors: Hak-Sung Kim, Daejeon (KR); Hee-Sung Park, Gwangiu (KR); Sung-Hun Nam, Gyeonggi-do (KR); Jin-Hyun Kim, Seoul (KR)

Correspondence Address: STAAS & HALSEY LLP, SUITE 700, 1201 NEW YORK AVENUE, N.W., WASHINGTON, DC, 20005, US

(21) Appl. No.: 12/085,672

(22) PCT Filed: Nov. 28, 2006
(86) PCT No.: PCT/KR2006/005046

§ 371 Date: May 29, 2008

(30) Foreign Application Priority Data
Dec. 5, 2005 (KR) 10-2005-0117353

(51) Int. Cl. C40B 10/00; C07K 2/00

(52) U.S. Cl. 530/350; 506/1

(57) ABSTRACT

Disclosed is a method for preparing a protein having a new function. The method comprises (A) a functional element-designing step of designing functional elements required for a new function desired to impart to an existing protein scaffold; (B) a functional element-inserting step of simultaneously inserting at least two gene fragments corresponding to the designed functional elements into a protein scaffold gene; and (C) a mutant screening and improving step of screening a mutant having a new function from a library of mutants inserted with the mutant genes, and improving and optimizing the function of the screened mutant using a directed evolution technique. The method for preparing can be widely used for the development of therapeutic proteins and the creation of industrial enzymes in the fields of bioengineering and biotechnology.

### CROSS REFERENCE TO RELATED APPLICATIONS

This application claims the benefit under 35 U.S.C. Section 371, of PCT International Application Number PCT/KR2006/005046, filed Nov. 28, 2006, which claimed priority to Korean Application No. 10-2005-0117353, filed Dec. 5, 2005 in Republic of Korea, the contents of which are incorporated herein by reference.

### SEQUENCE LISTING

This application contains a Sequence Listing which has been submitted and is hereby incorporated by reference in its entirety. The ASCII copy of the Sequence Listing, created Oct. 26, 2009, is named 19251002.tex, and is 20,283 bytes in size.

### TECHNICAL FIELD

The present invention relates to a method for preparing a protein having a new function, and more particularly to a method for preparing a protein having a new function in an existing protein scaffold by mimicking the natural evolution process.

### BACKGROUND ART

Proteins have been widely used for medical, therapeutic and industrial applications. However, most proteins have a limitation in that they are not easy to use by human beings due to their inappropriate properties, including stability, activity, specificity, and substrate specificity etc. To overcome this limitation, studies on proteins having desired properties and new functions have been continuously conducted.

To improve the properties of proteins, including folding, stability, activity, substrate specificity and ligand affinity, or prepare proteins having new functions, studies on rational design based on the structural information of proteins, the three-dimensional structure of which was found, and studies on directed evolution methods comprising randomly mutating protein-encoding genes and screening mutants having improved characteristics at high speed, have mainly been conducted. However, most of such prior techniques depend on the accumulation of single amino acid modifications caused by point mutations, and thus are methods of improving the properties of existing proteins, rather than preparing proteins having new functions.

Recently, the results have been reported which show that a ribose-binding protein having no catalytic function was imparted with a triose phosphate isomerase activity as a new catalytic function using computational protein design algorithms (Dwyer, M. A., Looger, L. L., and Hellinga, H. W., 2004, Science, 304, 1967). This method has an advantage of making a protein having a new function using computer algorithms, but it has a limitation in that the new catalytic function is imparted through calculations for partial modifications resulting from the point mutations of important amino acid sites.

To prepare proteins having new functions, knowledge on the correlation between the structures and functions of a larger number of proteins, and understanding on the natural evolution process of proteins, are further required, and new methods that reflect these requirements are required. Various proteins produced in the natural evolution process have been found to be produced through complex processes, including the modification of base sequences of existing genes, and the insertion, deletion and recombination of gene fragments having any lengths or base sequences over a long period of time. Also, protein scaffolds that maintain the structures of proteins are limited in number, and thus even in the case of proteins that perform different functions, the scaffolds thereof are frequently similar or equal to each other. Accordingly, to create proteins having targeted functions, a new technique capable of accepting such complex processes of proteins is required, and to create targeted functions in existing protein scaffolds, a process of redesigning the structure of active sites is required.

### DISCLOSURE

### Technical Problem

The present invention has been made in order to solve the above-described problems occurring in the prior art, and it is an object of the present invention to provide a method for efficiently preparing proteins having new functions, which mimics the natural evolution process of proteins.

Another object of the present invention is to provide a general technique capable of preparing various proteins having new functions using existing protein scaffold.

Still another object of the present invention is to create a new protein having metallo β-lactamase activity as a new catalytic function imparted to a human glyoxalase II scaffold using said method for preparing proteins having new functions.

### Technical Solution

To achieve the above objects, the present invention provides a method for preparing a protein having a new function, the method comprising: (A) a functional element-designing step of designing functional elements required for a new function desired to impart to an existing protein scaffold; (B) a functional element-inserting step of simultaneously inserting at least two gene fragments corresponding to the designed functional elements into a protein scaffold gene; and (C) a screening and improving step of mutants having a new function from a library of mutants inserted with the new gene segments, and improving and optimizing the function of the screened mutant using a directed evolution technique.

FIG. 1 schematically shows the method for preparing a protein having a new function according to the present invention. Hereinafter, each step of the preparation method according to the present invention will be described in detail.

(A) Design of Functional Elements Required for New Function

This is a step of designing functional elements required for a new function to be introduced, based on information on the structure and function of proteins.

First, a protein scaffold to be imparted with a new function is selected through the three-dimensional structure database and research literatures of existing proteins. To design active sites having a new function, it is advantageous to select a protein scaffold having a structure similar to a protein having a function to be created, rather than selecting a protein scaffold having a great structural difference.

As the scaffold of the target protein is determined, functional elements required for a new function to be inserted into the scaffold are selected and designed. Elements required for performing a new function in a protein, including catalytic elements constituting active sites, or important sites, such as loops (e.g., substrate binding sites and ligand binding sites) and amino acid fragments, are designed through the comparison of the similarity between the amino acid sequences of proteins, and through information on reaction mechanisms and three-dimensional structures.

In the catalytic elements, substitutions of specific amino acids necessary for a new function, such as amino acids that act directly on catalytic functions, or amino acids that coordinate or stabilize metals required for catalytic reactions, and loops and amino acid sites to be removed, which are unnecessary for or interfere with a new function, are selected. Such functional elements include single amino acids, and also substitutions and insertions of relatively long portions, such as amino acid fragments and protein secondary structures, and thus have a significant effect on the structure and function of the protein scaffold.

Among portions to be substituted, sites important for a new function are designed into mutant amino acid sequences having various lengths and sequences by comparatively analyzing the amino acid sequences of similar proteins, such that they include consensus amino acid sequences and random amino acid sequences. When synthetic genes corresponding to the designed functional elements are simultaneously introduced into a protein scaffold gene by PCR, the random sequences are inserted with random amino acids, such that a possibility of imparting a new function is increased.

(B) Simultaneous Incorporation of Designed Functional Elements by Gene Recombination

This is a step in which at least two mutant gene fragments corresponding to the designed functional elements are inserted into an existing protein scaffold gene through gene recombination by PCR.

First, portions unnecessary for a new function, which prevent the entry of new substrates and ligands or cause spatial limitations, are removed from the scaffold of the corresponding protein by gene recombination. Elements necessary for a new function, for example, amino acids that are directly involved in catalytic reaction or interaction, or amino acids that coordinate or stabilize metals to facilitate catalytic reactions, are substituted into the corresponding amino acids through gene mutation using overlapping extension PCR.

Into the protein scaffold, which was substituted with specific amino acids and from which the unnecessary portions were removed, functional elements, including amino acid fragments having relatively long length, and protein secondary structures, are simultaneously inserted. Protein secondary structures, such as amino acid fragments or loops that impose spatial restrictions on carrying out a new function or are unnecessary, are substituted with functional elements, including new amino acid fragments or protein secondary structures, such as substrate binding sites and ligand interaction sites. These functional elements are designed to have various lengths and sequences, including consensus sequences and random sequences, in order to efficiently create a desired function, and are inserted into the corresponding protein scaffold using a random or combinatorial method. For this purpose, synthetic oligonucleotides corresponding to various kinds of amino acid fragments, each including the amino acids of the consensus sequence and the amino acids of the random sequence, are synthesized, and various gene fragments having the respective single-functional elements including mutations are amplified by PCR.

The amplified gene fragments are purified, the genes corresponding to two or more functional elements are combined with each other, and recombined into a full-length mutant gene comprising a variety of all functional elements, by one-step PCR with primers having base sequences corresponding to both terminal ends of the protein scaffold gene, using the terminal sequence homology of the gene fragments. Also, in the PCR process, the reaction conditions are regulated such that mutations are induced in the entire gene, whereby the change in the function of a new protein is efficiently induced by additional mutations. Thus, a new function can be efficiently created by inducing a great change in amino acid sequences in important sites and inducing low mutations in other sites that are difficult to predict. Through such a series of gene recombination processes, functional elements required for active sites having a new function are simultaneously inserted into the corresponding protein scaffold using the random or combinatorial method, thus making a library of diverse mutants.

(C) Screening of Mutant and Improvement of Function Thereof Through Directed Evolution

This is a step of screening a mutant from the mutant library and stabilizing and improving the new function of the screened mutant through a directed evolution method.

Both terminal ends of the full-length mutant gene obtained by PCR in the step (B) are treated with restriction enzymes. The treated gene is cloned into a plasmid and transformed into a bacterial strain such as E. coli, thus making a library. From the library, mutants having a new function are screened by measuring a targeted function, such as catalytic activity, ligand affinity, or specificity using methods of measuring viability, activity, binding to ligand, fluorescence, etc.

Screened mutants having a new function mostly have very low activity or an unstable structure. Thus, to improve and stabilize the new function of the screened mutants, the properties of the mutants are improved by inducing mutations at specific gene sites using a directed evolution method effective for improving the properties of proteins. To improve the activity of the mutants, a method such as error prone-PCR or DNA shuffling is mainly used.

Through a series of processes as described above, a new protein having a new function in a protein scaffold can be effectively prepared.

### ADVANTAGEOUS EFFECTS

As described above, according to the present invention, a variety of proteins having a targeted function can be efficiently created by designing functional elements required for the new functions through information on existing proteins, inserting the designed elements into the scaffolds of the proteins and carrying out the directed evolution of the proteins.

### DESCRIPTION OF DRAWINGS

FIG. 1 schematically shows a protein having a new function according to the present invention.

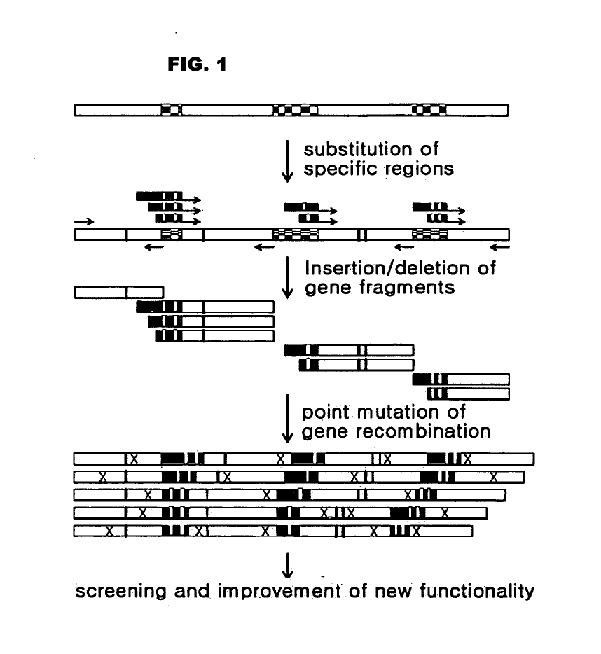

FIG. 2 is a view of protein structures, which shows a process of introducing new metallo β-lactamase activity into a glyoxalase II scaffold.

FIG. 3 shows that the amino acid sequences of substrate binding sites required for introducing metallo β-lactamase catalytic activity are designed through the comparison of sequences between similar proteins, such that they include consensus sequences and random sequences. FIG. 3 discloses the loop1 sequences as SEQ ID NOS 1 and 30-33, respectively, in order of appearance. Loop2 sequences disclosed as SEQ ID NOS 2 and 34-37, respectively, in order of appearance. Loop4 sequences disclosed as SEQ ID NOS 3 and 38-40, respectively, in order of appearance. Loop6 sequences disclosed as SEQ ID NOS 4-6 and 41-44, respectively, in order of appearance.

FIG. 4 is a schematic diagram showing the portions of a glyoxalase II scaffold, which are to be inserted with the designed substrate binding sites.

FIG. 5 shows the amino acid sequence of a mutant having new metallo β-lactamase catalytic activity and showing the highest activity, together with the amino acid sequences of glyoxalase II (SEQ ID NOS 45 and 46, respectively, in order of appearance), β-lactamase (IMP-1) (SEQ ID NO:47) and evMBL8 (SEQ ID NO: 29).

### BEST MODE

According to present invention, the usefulness of the present invention was confirmed by preparing a new protein in which metallo β-lactamase activity as a new catalytic function was imparted to a glyoxalase II scaffold isolated from human beings, according to the above-described method.

Human glyoxylase II and Pseudomonas aeroginosa metallo β-lactamase show a low amino acid similarity (about 13%) therebetween, but have significantly similar structures and the same protein scaffold. However, they perform different catalytic reactions. For this reason, in order to impart new metallo β-lactamase activity to the glyoxalase II scaffold, the redesign of active sites and the introduction of substrate binding sites should be performed.

Hereinafter, the present invention will be described in detail with reference to examples of imparting metallo β-lactamase activity as a new catalytic function to a glyoxalase II scaffold isolated from human beings. It is to be understood, however, that these examples are for illustrative purposes only, and the scope of the present invention are not limited thereto.

### EXAMPLES

### Introduction of Metallo β-Lactamase Activity into Glyoxalase II Scaffold

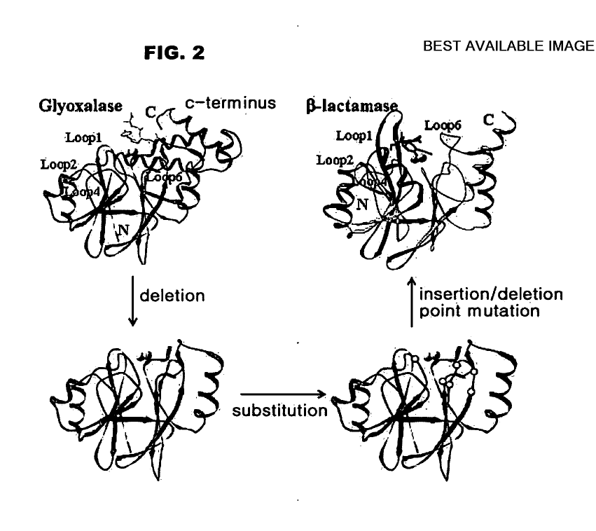

An overall process of imparting new metallo β-lactamase (IMP-1) activity to a glyoxalase II (GlyII) scaffold is shown in FIG. 2. Hereinafter, each of the process will be described in detail.

### Example 1

### Design of Functional Elements for Imparting Metallo β-Lactamase

Functional elements required for a new function were designed through information on the amino acid sequences and three-dimensional structures of the above-described two proteins and through the comparative analysis of the sequences of similar proteins.

1) Design of Functional Elements by Analysis of Spatial Arrangement

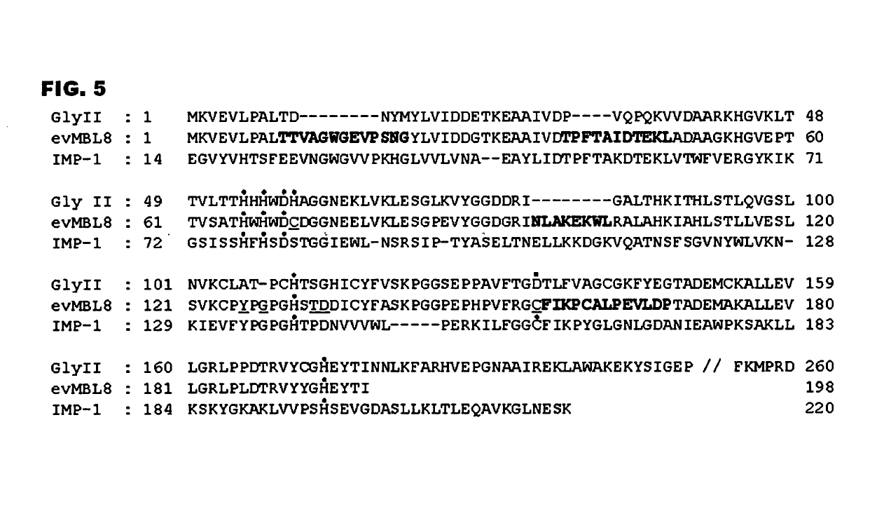

The sequences of glyoxalase II and metallo β-lactamase are shown in FIG. 5.

Glyoxalase II has an amino acid length longer than that of metallo β-lactamase and contains a glutathione binding domain (amino acids 178-260) as an original substrate at the C-terminal region. This domain spatially restricts the binding of glyoxalase II to β-lactam antibiotics such as cefotaxime as the substrate of metallo β-lactamase to be newly imparted. In order for glyoxalase II to efficiently bind to β-lactam antibiotics so as to have a function of metallo β-lactamase, it is preferable to remove the C-terminal region.

2) Design of Amino Acids by Analysis of Amino Acids Required for Coordination and Stabilization of Metals at the Active Site.

Then, amino acids required for the coordination and stabilization of metals involved in catalytic mechanisms in metallo β-lactamase were analyzed. In metallo β-lactamase, two zincs are involved in the catalytic mechanisms, in which zinc 1 is coordinated by His77, His79 and His139 amino acids, and zinc 2 is coordinated by Asp81, Cys158 and His197 amino acids. In glyoxalase II, amino acids involved in the coordination of zinc 1 are substantially the same as those of metallo β-lactamase, but in the case of zinc 2, Cys158 amino acid is substituted with Asp134, and His59 amino acid is additionally involved in coordination. Thus, in order to insert metal-coordination elements required for imparting a metallo β-lactamase function to a glyoxalase II scaffold, it is preferable to substitute His59 and Asp134 amino acids with Cys.

In metallo β-lactamase, amino acids such as Gly159, Thr164 and Asp165 are known to function to coordinate metals in a correct direction by the metal-coordinating amino acids through interaction such as hydrogen binding to amino acid reactive groups around active sites (Scrofani et al., 1999, Biochemistry, 38, 14507). Thus, when Gly is inserted between Thr107 and Pro108 in the glyoxalase II scaffold, and Ser112 and Gly113 are substituted with Thr and Asp, respectively, it is expected that the stable coordination of metals can be induced.

3) Design of Functional Elements by Structural Analysis of Substrate Binding Sites

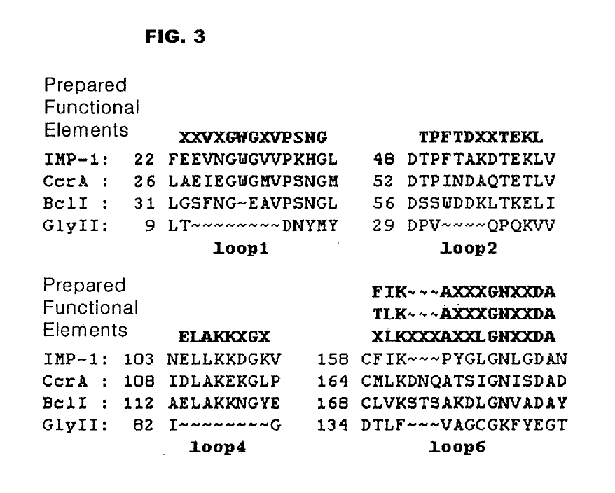

The substrate binding sites of two proteins show a great structural difference therebetween. In the case of metallo β-lactamase, loops 1, 2, 4 and 6 perform an important role in binding to β-lactam antibiotics and catalytic reactions. Particularly, Lys161 and Asn167 of loop 6 and Lys107 and Lys108 of loop 6 perform an important role in binding to substrates and catalytic reactions. To insert loop elements required for binding to substrate β-lactam antibiotics into a glyoxalase II scaffold, amino acid sequences corresponding to the metallo β-lactamase proteins of P. aeruginosa (IMP-1), Bacteriodes fragilis (CerA) and Bacillus cereus (BcII), which are similar to each other in terms of the theory of evolution, are shown and compared to each other in FIG. 3. The functional elements were designed such that the important site and consensus site of each of the amino acid sequences were maintained intact while the remaining sites contained random amino acids, whereby a function could be more easily acquired. The designed functional elements are as follows:

Loop 1:

(SEQ ID NO: 1)

Xaa Xaa Val Xaa Gly Trp Gly Xaa Val Pro Ser Asn
Gly;
Loop 2:

(SEQ ID NO: 2)

Thr Pro Phe Thr Asp Xaa Xaa Thr Glu Lys Leu;
Loop 4:

(SEQ ID NO: 3)

Glu Leu Ala Lys Lys Xaa Gly Xaa;
Loop 6:

(SEQ ID NO: 4)

1) Phe Ile Lys Ala Xaa Xaa Xaa Gly Asn Xaa Xaa
Asp Ala;

(SEQ ID NO: 5)

2) Thr Leu Lys Ala Xaa Xaa Xaa Gly Asn Xaa Xaa
Asp Ala;
and

(SEQ ID NO: 6)

3) Xaa Leu Lys Xaa Xaa Xaa Ala Xaa Xaa Leu Gly
Asn Xaa Xaa Asp Ala.

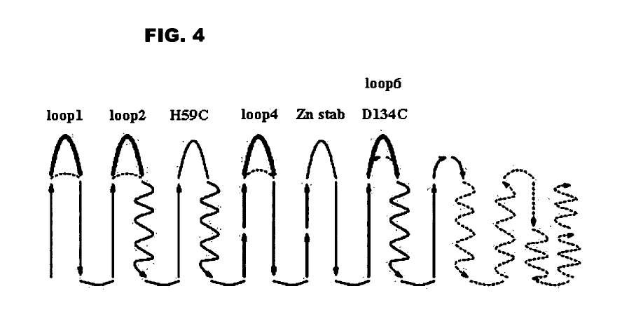

Because loop 6 shows a severe variation in amino acids among similar family proteins and is a functionally important site, it was designed with three different amino acid sequences, and Xaa was designed to include random amino acids. A glyoxalase II scaffold domain to be inserted with each of the functional loops is shown in FIG. 4.

### Example 2

### Insertion of Gene Fragments for Introducing Metallo β-Lactamase Functional Elements into Glyoxalase II Scaffold

1) Introduction of Functional Elements by Analysis of Spatial Arrangement

Based on the results of design of functional elements in Example 1-1, the C-terminal region including the glutathione binding domain of glyoxalase II was removed through gene recombination by PCR.

Specifically, the corresponding gene fragment was amplified by PCR (5 min at 94° C.; 30 cycles of 1 min at 94° C., 30 sec at 55° C., and 30 sec at 72° C.; and then 5 min at 72° C.) using a glyoxalase II gene as a template, an N-terminal primer (SEQ ID NO: 7) having an EcoRI cleavage site, and a C-177 terminal primer (SEQ ID NO: 8) having a HindIII cleavage site.

SEQ ID NO: 7:
5′ -CCCGAATTCATGAAGGTAGAGGTGCTG-3′
SEQ ID NO: 8:
5′ -CCCAAGCTTTTAGATGGTGTACTCGTGGCC-3′

The both terminal ends of the amplified glyoxalase II gene fragment were digested with EcoRI and HindIII, and cloned into pMAL-p2k having a kanamycin-resistant gene digested with the same restriction enzymes. The pMAL-p2k was a vector obtained by amplifying the gene fragment (except for an ampicillin-resistant gene fragment) of an existing pMAL-p2x (New England Biolabs) by PCR (5 min at 94° C.; 30 cycles of 3 min at 94° C., 3 min at 55° C. and 3 min at 72° C.; and then 5 min at 72° C.) using primers of Nmal (SEQ ID NO: 9) and Cmal (SEQ ID NO: 10), treating the amplified gene fragment with restriction enzyme kpnI together with a kanamycin-resistant gene amplified from pACYC177 (New England Biolabs) by PCR (5 min at 94° C.; 30 cycles of 1 min at 94° C., 30 sec at 55° C. and 30 sec at 72° C.; and then 5 min at 72° C.) using primers of Nkan (SEQ ID NO: 11) and Ckan (SEQ ID NO: 12), and cloning the genes. The cloned vector was transformed into expression strain E. coli XL1-Blue, and the sequence was confirmed, thus obtaining a glyoxalase II scaffold from which the C-terminal region unnecessary for a new function was removed.

SEQ ID NO: 9:
5′ -CCCGGTACCGGGCGCGTAAAAGGATCT-3′;
SEQ ID NO: 10:
5′ -CCCGGTACCTCGACTGAGCCTTTCGTT-3′;
SEQ ID NO: 11:
5′ -GCGGGTACCTGATCTGATCCTTCAACT-3′;
SEQ ID NO: 12:
5′ -GCGGGTACCCTCAGCAAAAGTTCGATT-3′

2) Introduction of Functional Elements by Analysis of Amino Acids Required for Immobilization and Stabilization of Metals

The glyoxalase II scaffold, from which the C-terminal region was removed in the above section 1), was substituted with amino acids required for the coordination and stabilization of metals in catalytic mechanisms in the metallo β-lactamase designed in Example 1-2. Specifically, for the introduction of metal coordination factors into the glyoxalase II scaffold, His59 and Asp134 amino acids were substituted with Cys, and for the stabilization of metals, Gly amino acid was inserted between Thr107 and Pro108, and Ser112 and Gly113 amino acids were substituted with Thr and Asp, respectively, through gene recombination by PCR.

Specifically, the forward region of the C-terminal mutated glyoxalase II scaffold gene prepared in the above step 1) was amplified PCR (5 min at 94° C.; 30 cycles of 1 min at 94° C., 30 sec at 55° C. and 30 sec at 72° C.; and then 5 min at 72° C.) using an N-terminal primer (SEQ ID NO: 7) having a restriction enzyme EcoRI cleavage site, and forward mutation-inducing primers (His→Cys, SEQ ID NO: 13; Asp134→Cys, SEQ ID NO: 14; Thr107Pro108˜Ser112Gly113→Thr107GlyPro108˜Thr112Asp113, SEQ ID NO: 15).

Also, the reverse region of the scaffold gene was amplified by PCR using a C-177 terminal primer (SEQ ID NO: 8) having a HindIII cleavage site and reverse mutation-inducing primers (His59→Cys, SEQ ID NO: 16; Asp134→Cys, SEQ ID NO: 17; Thr107Pro108˜Ser112Gly113→Thr107GlyPro108→Thr112Asp113; SEQ ID NO: 18) in the above PCR conditions.

SEQ ID NO: 13:
5′ -GTCCCAGTGGTGGTGGGT-3′
SEQ ID NO: 14:
5′ -ACCTGTGAACACGGCAGG-3′
SEQ ID NO: 15:
5′ -CAGGCACTTGACGTTCAG-3′
SEQ ID NO: 16:
5′ -ACCCACCACCACTGGGACTGTGCTGGCGGGAATGAG-3′
SEQ ID NO: 17:
5′ -CCTGCCGTGTTCACAGGTTGTACCTTGTTTGTGGCTGGC-3′
SEQ ID NO: 18:
5′ -CTGAACGTCAAGTGCCTGTATACCGGGCCGTG~
~CCACACTACAGACCACATTTGTTACTTCGTG-3′

The amplified forward and reverse gene fragments for each of mutant amino acids were purified on agarose gel, and combined with each other and subjected to overlapping PCR (5 min at 94° C.; 30 cycles of 1 min at 94° C., 1 min at 55° C. and 1 min at 72° C.; and then 5 min at 72° C.) using the N-terminal primer (SEQ ID NO: 7) having a restriction enzyme EcoRI cleavage site and the C-177 terminal primer (SEQ ID NO: 8) having an HindIII cleavage site, thus obtaining a gene having mutations which occurred in the respective amino acids. The both terminal ends of the mutant gene were digested with restriction enzymes EcoRI and HindIII, and cloned into pMAL-p2k having a kanamycin-resistant gene digested with the same restriction enzymes. The cloned vector was transformed into expression strain E. coli XL1-Blue, and the base sequence of the gene was analyzed to confirm whether a mutation in the corresponding amino acid occurred. Through the above-described overlapping PCR method, a mutant glyoxalase II scaffold gene substituted with all the amino acids to be mutated was obtained.

3) Introduction of Designed Functional Elements by Structural Analysis of Substrate Binding Sites

Oligonucleotides encoding the amino acid sequences of the functional elements designed in Example 1-3 were synthesized as follows:

SEQ ID NO: 19:
5′-CAGGGCAGGCAGCACCTC-3′;
SEQ ID NO: 20:
5′-GAGGTGCTGCCTGCCCTGNNSNNSGTTNNSGGGT~
~GGGGCNNSGTACCTTCCAACGGGTACCTGGTCATTGATGAT-3′;
SEQ ID NO: 21:
5′-ATCCACAATGGCAGCCTC-3′;
SEQ ID NO: 22:
5′-GAGGCTGCCATTGTGGATACTCCATTTACGGATNN~
~SNNSACTGAAAAGTTAGTGGACGCGGCGAGAAAG-3′;
SEQ ID NO: 23:
5′-GATACGGTCGTCACCCCC-3′;
SEQ ID NO: 24:
5′-GGGGGTGACGACCGTATCGAGCTCGCCAAGAAAN~
~NSGGGNNSGGGGCCCTGACTCACAAG-3′;
SEQ ID NO: 25:
5′-ACCTGTGAACACGGCAGG-3′;
SEQ ID NO: 26:
5′-CCTGCCGTGTTCACAGGTTGTTTTATTAAAGCG~
~NNSNNSNNSGGCAATNNSNNSGACGCAACTGC~
~GGATGAGATGTGT-3′;
SEQ ID NO: 27:
5′-CCTGCCGTGTTCACAGGTTGTACCTTGAAAGCGN~
~NSNNSNNSGGCAATNNSNNSGACGCAACTGCGGATG~
~AGATGTGT-3′;
and
SEQ ID NO: 28:
5′-CCTGCCGTGTTCACAGGTTGTNNSTTGAAANNS~
~NNSNNSGCCNNSNNSTTGGGCAATNNSNNSGACGC~
~AACTGCGGATGAGATGTGT-3′.

The mutant gene fragments corresponding to the respective substituted loop domains were amplified by PCR using the following primer combinations: N-terminal primer (SEQ ID NO: 7)/loop 1-forward primer (SEQ ID NO: 19), loop 1-reverse primer (SEQ ID NO: 20)/loop 2-forward primer (SEQ ID NO: 21), loop 2-reverse primer (SEQ ID NO: 22)/loop 4-forward primer (SEQ ID NO: 23), loop 4-reverse primer (SEQ ID NO: 24)/loop 6-forward primer (SEQ ID NO: 25), loop 6-(1) reverse primer (SEQ ID NO: 26)/C-177 terminal primer (SEQ ID NO: 8)-loop 6(2) reverse primer (SEQ ID NO: 27)/C-177 terminal primer (SEQ ID NO: 8), loop 6-(3) reverse primer (SEQ ID NO: 28)/C-177 terminal primer (SEQ ID NO: 8).

For effective amplification, vent polymerase having high amplification accuracy was used, and said PCR reaction was performed in the following conditions: 5 min at 94° C.; 30 cycles of 1 min at 94° C., 30 sec at 55° C. and 30 sec at 72° C.; and 5 min at 72° C. Each of 7 mutant gene fragments obtained through the PCR reaction was purified on agarose gel, and the purified gene fragments were combined with each other and subjected to overlapping PCR in the following conditions using the N-terminal primer (SEQ ID NO: 7) and the C-177 terminal primer (SEQ ID NO: 8): 5 min at 94° C.; 35 cycles of 30 sec at 94° C., 30 sec at 50° C. and 30 sec at 72° C.; and 5 min at 72° C. Through the overlapping PCR, the mutant gene fragments containing the respective mutant loops were recombined, such that the designed mutant loops were simultaneously inserted through one-step PCR. In this recombination process, Taq polymerase having low accuracy was used, MnCl₂ and dNTP among reaction constituents were regulated to reduce amplification accuracy so as to induce gene mutations at random sites. For this purpose, PCR was performed in the following conditions: each gene fragment (˜1 pg), 1×Taq polymerase buffer (75 mM Tris-HCl, pH 8.8, 20 mM (NH₄)₂SO₄, 0.01% (v/v) Tween 20, 1.25 mM MgCl₂), dNTP (sATP and dGTP, 1.0 mM; dCTP and dTTP, 0.2 mM), 0.1-1.0 mM MnCl₂, 2.5 U of Taq polymerase, 100 pmol N-terminal primer (SEQ ID NO: 7) and C-177 terminal primer (SEQ ID NO: 8).

Both ends of the mutant genes containing all the designed elements, obtained through the above process, were digested with restriction enzymes EcoRI and HindIII and cloned into a pMAL-p2k vector having a kanamycin-resistant gene. The cloned vector was transformed into E. coli, thus constructing a library of mutants comprising functional elements having various amino acid sequences.

### Example 3

### Selection and Improvement of Mutant Having Metallo β-Lactamase Activity

From the library of diverse mutants, constructed in Example 2, mutants having a metallo β-lactamase catalytic function were selected through the viability of E. coli by a catalytic activity of degrading cefotaxime as a substrate β-lactam antibiotic.

First, E. coli was cultured in an LB solid medium containing 0.05 mM isopropyl-β-D-thiogalactoside (IPTG), 0.2 mM ZnCl₂, 50 mg/ml kanamycin, and 0.2 mg/ml cefotaxime, and E. coli colonies growing in the culture medium were selected. The growing colonies were finally selected through a two-step reselecting process comprising transferring the colonies into a fresh solid medium containing the same concentration of cefotaxime, growing colonies in the medium, isolating a plasmid containing the corresponding mutant gene in order to eliminate of E. coli itself, transforming the isolated plasmid into fresh E. coli, and screening colonies in the E. coli strain. From the library of 2×10⁷ mutants, obtained through the above-described recombination process using overlapping PCR, 13 active mutants were finally selected.

The analysis of the base sequences of the mutants showed that these mutants all contained the amino acid sequence of SEQ ID NO: 4 in the loop 6, and 2-9 random amino acid mutations occurred in the entire mutant genes. These active mutants had a very low catalytic activity, and the metallo β-lactamase activity thereof could not be measured through a method such as spectrophotometry or liquid chromatography. For this reason, in the next step, the activity of the mutants having metallo β-lactamase activity was increased using a directed evolution method.

Because the efficiency of the directed evolution method depends on the diversity of starting mutant genes, a larger number of mutants were secured. In the case of loop 6, only the amino acid sequence of SEQ ID NO: 4 was subjected again to overlapping PCR according to the above-described method to prepare a library of 1.5×10⁸ mutants, and 313 active mutants were finally selected through the same selecting and reselecting processes as described above. The activity of the selected active mutants was increased through the prior DNA shuffling method (Stemmer, W. P., 1994, Nature, 370, 389). The activity of the mutants was gradually increased from 0.2 mg/ml to 4.5 mg/ml through a seven-step DNA shuffling process, and 15 active mutants, which grew even at a cefotaxime concentration of 4.5 mg/ml, were selected.

Finally, a best mutant (evMBL8) showing the highest metallo β-lactamase catalytic activity was selected, and deposited in the Korean Collection for Type Cultures (KCTC), the Korean Research Institute of Bioscience and Biotechnology, on Dec. 2, 2005 under accession number: KCTC 10877BP). Also, the gene sequence of the mutant was examined by base sequence analysis, and the amino acid sequence (SEQ ID NO: 29) of the mutant is shown in FIG. 5 together with the sequences of glyoxalase II and metallo β-lactamase. It could be seen that the mutant (evMBL8) acquired new metallo β-lactamase catalytic activity through the mutations of 81 amino acids among 198 amino acids of an initial gloxalase II scaffold while it underwent a gene recombination process consisting of several steps.

To analyze the characteristics of the mutant (evMBL8), the mutant was cultured in an LB medium containing 50 mg/ml kanamycin, 0.1 mM IPTG and 0.2 mM ZnCl₂, and the cultured mutant was collected, and re-suspended in a 50 mM Hepes buffer (pH 7.4, 20 mM NaCl). The suspension was ultrasonically disrupted, and the supernatant was collected, and passed through amylose resin, thus purifying evMBL8 bound to a maltose-binding protein (MBP). The catalytic activity of the purified evMBL8 mutant protein was examined by adding the mutant to 1 ml of a mixture of 50 mM Hepes buffer (pH 7.4) and 0.02-2.0 mM cefotaxime and measuring a reduction in absorbance at 260 nm resulting from the degradation of the substrate cefotaxime, using a spectrophotometer. As a result, the evMBL8 mutant showed an activity of kcat/Km=about 1.8×10² M⁻¹S⁻¹ for the substrate cefotaxime.

Also, the cefotaxime resistance of an E. coli strain containing the evMBL8 mutant gene was examined. For this purpose, each of an E. coli strain containing the evMBL8 mutant gene, and an E. coli strain containing no evMBL8 mutant gene, was cultured at 30° C. in 5 ml liquid medium containing 0.05 mM IPTG, 0.2 mM ZnCl₂, 50 mg/ml kanamycin and varying concentrations (0.02-2.0 mg/ml) of cefotaxime, while the growth of the E. coli strains was analyzed with a spectrophotometer (OD₆₀₀) at two-hr intervals. As a result, it was observed that the E. coli strain containing the evMBL8 mutant showed a resistance to cefotaxime, which was at least 100 times higher than the E. coli strain having no metallo β-lactamase activity.

### INDUSTRIAL APPLICABILITY

As described above, according to the present invention, a variety of proteins having a targeted function can be prepared by designing functional elements required for the targeted function, through information on existing proteins, simultaneously inserting the designed functional elements into the existing proteins, subjecting the proteins to directed evolution.

The inventive method for preparing proteins having a targeted function can be widely used for the development of therapeutic proteins and the creation of industrial enzymes in the fields of bioengineering and biotechnology.

1. A method for preparing a protein having a targeted function comprising:
(A) a functional element-designing step of designing functional elements required for a new function desired to impart to an existing protein scaffold;
(B) a functional element-inserting step of simultaneously inserting at least two gene fragments corresponding to the designed functional elements into a protein scaffold gene; and
(C) a mutant screening and improving step of screening a mutant having a new function from a library of mutants inserted with the mutant genes, and improving and optimizing the function of the screened mutant using a directed evolution technique.

2. The method of claim 1, wherein the functional elements in the step (A) are either amino acid fragments containing consensus amino acid sequences and random amino acid sequences, or protein secondary structures.

3. The method of claim 1, wherein the step (B) of simultaneously inserting at least two gene fragments is performed by PCR-amplifying each of the gene fragments corresponding to at least two functional elements, purifying the amplified fragments, and PCR-amplifying each of the purified fragments with primers having base sequences corresponding to the both terminal ends of the protein scaffold gene, using the terminal sequence homology of the gene fragments, so as to recombine the gene fragments into a full-length gene such that the designed functional elements are simultaneously inserted into the protein scaffold gene.

4. The method of claim 1, wherein the screening of the mutant in the step (C) is performed by measuring catalytic activity, ligand affinity, or fluorescence, according to the targeted function of the protein.

5. The method of claim 1, wherein the directed evolution technique is error-prone PCR or DNA shuffling.

6. Mutant protein evMBL8 (accession number: KCTC 10877BP) of SEQ ID NO: 29, in which functional elements required for metallo β-lactamase activity are introduced into a glyoxalase II scaffold according to the method of claim 1.
